(19) United States
(12) Patent Application Publication
(10) Pub. No.: US 2018/0338897 A1
(43) Pub. Date: Nov. 29, 2018

(54) ORAL COMPOSITIONS FOR REDUCING DENTAL STAINS

(71) Applicant: New York University, New York, NY (US)

(72) Inventors: Charles P. Martucci, Scarsdale, NY (US); Khagay Nagdimov, Brooklyn, NY (US)

(21) Appl. No.: 15/988,810

(22) Filed: May 24, 2018

Related U.S. Application Data
(60) Provisional application No. 62/510,452, filed on May 24, 2017.

(51) Int. Cl. A61K 8/49; A61K 8/02; A61K 8/04; A61Q 11/00

(52) U.S. Cl. CPC A61K 8/4926; A61K 8/022; A61Q 11/00; A61K 8/0216; A61K 8/042

(57) ABSTRACT

Disclosed are compositions and formulations for reducing or removing tooth stains. The composition comprise one or more compounds having the formula

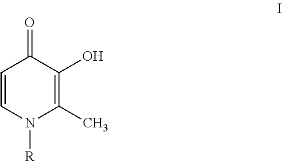

where R is

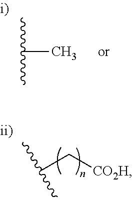

where n=1 to 11, in an orally acceptable carrier. Also disclosed are methods of making and use the compounds in oral formulations.

### CROSS REFERENCE TO RELATED APPLICATIONS

The application claims priority to U.S. Provisional Application No. 62/510,452, filed on May 24, 2017, the disclosure of which is hereby incorporated by reference.

### FIELD OF THE DISCLOSURE

The disclosure generally relates to compositions and methods for reducing or removing tooth stains.

### BACKGROUND OF THE DISCLOSURE

Dental treatments for aesthetical purposes in the United States have become very popular. Sales of at-home bleaching products exceed $1 million annually. However, some of the products have undesirable side effects. For example, teeth whitening solutions such as carbamide peroxide have a negative effect on the surface enamel. Bleaching is considered to induce surface morphological changes, decrease enamel micro-hardness, and reduce fracture toughness. These changes provide a permissive environment for subsequent staining of the teeth. For this reason, after a bleaching procedure, individuals are advised to avoid beverages that cause tooth staining such as red wine, tea, cola, and black coffee.

Staining from beverages mainly occur due to tannins. Tannins attach to the enamel of a human tooth. When the enamel is compromised by a bleaching treatment, it becomes easier for the tannins to attach to it. While ethylenediaminetetraacetic acid (EDTA) is known to reduce stains, its calcium chelating effects can be detrimental to the teeth. As a result, there is a continued need for better teeth stain removal approaches.

### SUMMARY OF THE DISCLOSURE

The present disclosure provides oral compositions, which are useful for removal or reduction of stains from teeth surfaces. The compositions comprise one or more compounds of Structure I in a formulation suitable for use in the oral cavity.

The oral compositions comprise a compound of the following structure (Structure I):

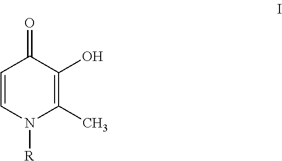

where R is

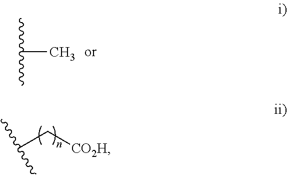

where n=1 to 11, in an orally acceptable carrier.

Examples of specific compounds include, deferiprone (R is CH₃; 3-hydroxy-1,2-dimethylpyridin-4(1H)-one), C-2 (where R is

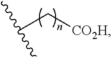

and n=1; 1-Carboxymethyl-3-Hydroxy-2-Methyl-Pyrid-4-one), C-6 (where R is

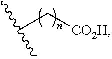

and n=5; 1-(6′-Carboxypentyl)-3-Hydroxy-2-Methyl-4-Pyridinone), C-11 (where R is

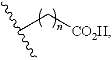

and n=10; 1-(11′-Carboxydecyl)-3-Hydroxy-2-Methyl-4-Pyridinone)), and C-12 (where R is

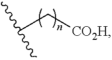

and n=11; 1-(12′-Carboxyundecyl)-3-Hydroxy-2-Methyl-4-Pyridinone)). The one or more compounds of Structure I are present in the formulations at concentration of from 0.001 to 1.0 M.

The oral compositions disclosed herein can be applied directly to teeth. For example, the compositions can be used in the form of gel, paste, tooth powder, mouth rinse, lozenges, chewing gum, dental strips and the like. The use of these compositions can be useful for reduction or removal of tooth stains.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1 shows destaining results.

FIG. 2 shows destaining results.

FIG. 3 shows sand white quartz and the silicon beads solutions.

FIG. 4 shows vials after iron (III) chloride, tannic acid, MOPS, and beads or sand white quartz was added. The rightmost vial is A1 and the vial numbers increase from left to right.

FIG. 5 shows control, 0.1 M and 0.01 M Deferiprone solutions from left to right.

FIG. 6 shows vials A2, B2, A3, B3, A4, B4 from left to right. Image is of solutions 10 minutes after they were prepared.

FIG. 7 shows vials A2, B2, A3, B3, A4, B4, A5, B5, B6 from left to right. Image is 10 minutes after A2-A4 and B2-B4 were prepared and 5 minutes after A5-A6 and B5-B6 were prepared.

FIG. 8 shows a growth curve of yeast over 24 hours. The absorbance was measured at 400 nm and plotted as a function of time.

FIG. 9 shows an inhibition of yeast growth on agar plates with disks loaded with EDTA, Deferiprone, C-2, and C-6. The control disk in the center of the plate. The circles were drawn around EDTA, Deferiprone, C-2, and C-6 disks to help visualize zones of antimicrobial activity.

FIG. 10 shows an inhibition of yeast growth on agar plates with disks loaded with EDTA, Deferiprone, C-2, and C-6. The control disk in the center of the plate. The circles were drawn around EDTA, Deferiprone, C-2, and C-6 disks to help visualize zones of antimicrobial activity.

### DESCRIPTION OF THE DISCLOSURE

Although claimed subject matter will be described in terms of certain embodiments/examples, other embodiments/examples, including embodiments/examples that do not provide all of the benefits and features set forth herein, are also within the scope of this disclosure. Various structural, logical, and process step may be made without departing from the scope of the disclosure.

Ranges of values are disclosed herein. The ranges set out a lower limit value and an upper limit value. Unless otherwise stated, the ranges include all values to the magnitude of the smallest value (either lower limit value or upper limit value) and ranges between the values of the stated range. As an illustrative example, any range provided herein includes all values that fall within the ranges to the tenth decimal place, unless indicated otherwise.

The present disclosure provides oral compositions, which are useful for removal or reduction of stains from teeth surfaces. The compositions comprise one or more compounds of Structure I in a formulation suitable for use in the oral cavity.

The oral compositions comprise a compound of the following structure (Structure I):

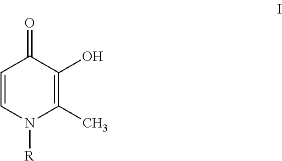

where R is

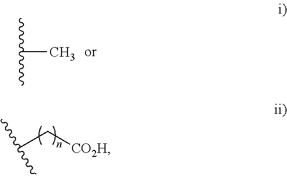

where n=1 to 11, in an orally acceptable carrier.

Examples of specific compounds include, deferiprone (R is CH₃; 3-hydroxy-1,2-dimethylpyridin-4(1H)-one), C-2 (where R is

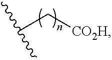

and n=1; 1-Carboxymethyl-3-Hydroxy-2-Methyl-Pyrid-4-one), C-6 (where R is

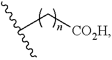

and n=5; 1-(6′-Carboxypentyl)-3-Hydroxy-2-Methyl-4-Pyridinone), C-11 (where R is

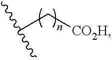

and n=10; 1-(11′-Carboxydecyl)-3-Hydroxy-2-Methyl-4-Pyridinone)), and C-12 (where R is

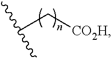

and n=11; 1-(12′-Carboxyundecyl)-3-Hydroxy-2-Methyl-4-Pyridinone)). The one or more compounds of Structure I are present in the formulations at concentration of from 0.001 to 1.0 M.

The compounds of this disclosure can be dissolved in water or other solvent systems may be used. For example, ethanol-water mixture or other solvents can be used.

The compounds of the present disclosure can reduce stains from the surfaces of teeth. The present compounds may also have anti-microbial activity. It was observed that C-2, C-6 at 0.001 M, and Deferiprone at 0.003 M inhibited the growth of yeast (Saccharomyces cerevisiae) when prepared with dextrose-agar plates. Thus, the compounds and compositions of the present disclosure may be used for reduction of teeth stains and/or as antimicrobials.

Deferiprone is available commercially. The other compounds may be synthesized using the general procedure described in Santos et al., J. Inorganic Biochem. 92(2002), 43-54, incorporated herein by reference. For example, C-11 was synthesized as described in Example 1.

The oral composition disclosed herein can be used for a human or an animal subject. The formulations can be applied directly to teeth in the form of strips and the like, or can be in the form of gel, paste, tooth powder, mouth rinse, lozenges, gums and the like. Selection of specific carrier components is dependent on the desired product formulation, as in known in the art.

Orally acceptable carrier materials can include solid or liquid excipients or diluents. The carrier material may comprise up to about 95% or more of the weight. Examples of orally acceptable carrier materials include water, glycerin, sorbitol, polyethylene glycols, propylene glycol and other edible polyhydric alcohols, ethanol and the like. One or more of viscosity modifiers, diluents, surface active agents, such as surfactants, emulsifiers, and foam modulators, pH modifying agents, abrasives, humectants, emollients, moisturizers, mouth feel agents, sweetening agents, flavor agents, and preservatives may also be included.

Surfactants may be anionic, nonionic, amphoteric, zwitterionic, cationic, or combinations thereof.

If a thicker formulation is desired (such as, for example, a gel or a paste), the composition may further comprise thickening or gelling agents. Examples include natural and synthetic gums and colloids. Natural gums include carrageenans, tragacanth, xanthan gum and the like. Synthetic thickeners include polyglycols and cellulose polymers such as hydroxyethyl cellulose and hydroxpropyl cellulose. Other inorganic thickeners include natural and synthetic clays such as hectorite clays, lithium magnesium silicate (laponite) and magnesium aluminum silicate (Veegum).

In certain embodiments, a fluoride source may be added to the oral compositions. Fluoride source compounds are well known the field of oral compositions.

The compositions may include one or more antimicrobial agents. Examples include quaternary ammonium compounds effective for providing anti-bacterial effect.

Flavors can be present in an amount of 0.1% to 3% by weight of the oral care composition. Examples of some flavors and flavoring materials used in oral care compositions are mint oils, wintergreen, clove bud oil, cassia, sage, parsley oil, marjoram, lemon, orange and the like.

The compositions may also contain added artificial colors. The color(s) can be any desired color, including blue, green and the like.

The compositions can be used as needed or on a regular basis. They may be used once a day or multiple times a day.

The invention is further described through the following examples, which are intended to be illustrative, and not restrictive, thus they are not intended to be limiting in any matter.

### Example 1

### 1. Preparation of 1-(11′-Carboxydecyl)-3-Hydroxy-2-Methyl-4-Pyridinone

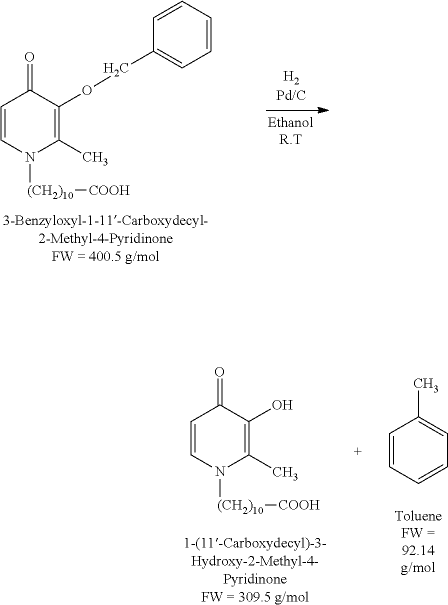

50 mL of 95% ethanol was added to a flask, followed by 0.2379 g of BCMP-11 (called I-55f crop). The mixture was capped and briefly shaken. 99 mg Pd/C was added to the mixture forming a reaction mixture. The flask was wrapped in bubble wrap and placed in a metal can to minimize flammability and bumping.

A Parr Hydrogenation Apparatus was used to remove the protecting benzyl group. Air was removed from the tank by vacuum and H₂ was added.

The reaction mixture was mixed (e.g., on a shaker) at room temperature for 20 hours.

Cooling was effected to crystallize the product by placing the reaction mixture in an ice machine at 0° C. The resulting crystals were called precipitate I-81a crop. In certain examples, the catalyst was removed via filtration prior to isolation of the precipitate I-81a.

If a product crystallized prior to removal of the catalyst, the reaction mixture containing the catalyst and crystallized product was heated to solubilize the crystallized product. After solubilizing the crystallized product, the catalyst was removed via filtration, and the filtrate was collected. The catalyst was then rinsed with ethanol and the ethanol used for rinsing was added to the filtrate.

The isolated filtrate combined with the ethanol used for rinsing was placed in a 500 mL round bottom flask and the solvent was removed via rotary evaporation (e.g., using a rotary evaporator with a bath temperature of 40° C.). Product net weight after this step was 0.2284 g. The product was dried under vacuum for 4 hours at room temperature. The weight was 0.1900 g (now termed I-81c). A sample of I-81c was used for seed crystals.

TLC plate Chromatography. The purpose of this was to determine the purity of the product and to test for iron binding. Solvent used was n-Butanol-HOAc—H₂O (80:20:20). Samples were BCMP-11 starting material, I-81b, and C-6 (I-22b). The TLC plate was analyzed under UV light.

To recrystallize the product, 3.0 mL ethanol was added to I-81c sample in the round bottom flask. Crystals were partially resuspended. The color was light black. The flask was placed in a hot water bath. A stream of N₂ was introduced into the flask. Partial crystallization occurred with much larger crystals. 6 mL ethanol was added to the flask. The flask was placed in the hot water bath again, and then allowed to cool under the hood. The color of the contents were now blacker in color. The flask was allowed to stand for 25 minutes. At this point there were still no crystals so the flask was placed in a 4° C. refrigerator. Two days later the sample was placed in vacuum filtration apparatus. The remainder was washed in a flask with 2 mL ethanol and poured into filtration funnel. Product was collected on filter paper, placed under a hood to dry. The filtrate was collected and refrigerated. The product was labeled I-83a and the product weight was 0.0876 g. Vacuum filtration was repeated with mother liquor (from round bottom flask) and 2nd crop from fridge. The drying was repeated as above. The collected sample was called I-83b. Melting point was determined using Fisher Melting Point Apparatus. The melting point for I-83a was 168-170° C., and for I-83b was 166-167° C.

C-2, C-6 and C-12 were synthesized by a similar process. The melting point for C-2 was 262° C.; for C-6 was 220-225° C.; for C-11 was 168-170° C.; and for C-12 was 170-172° C. The NMR spectra for all of the compounds was consistent with their proposed structures.

### Example 2

To simulate the effects of staining caused by coffee and tea, tannic acid and iron(III) chloride solutions were prepared.

To prepare the tannic acid solution, solid tannic acid was used. 0.1M FeCl₃.6H₂O and added to 100 mL of distilled water. 0.1 M solutions of MOPS buffer was prepared. Then 0.1M solutions of EDTA in distilled water, C-2 in MOPS, C-6 in MOPS, and deferiprone in MOPS, were prepared. For a tooth like structure, boiling beads made from synthetic silicate were used.

Methods were as follows: Experiment #1: Comparison of EDTA, C-2, and deferiprone destaining: The procedure was broken down into 2 phases: staining and destaining. In the staining phase, 7 beads were added to each of the 12 vials. 3 vials were labeled control, 3 were labeled EDTA, 3 were labeled C-2, and 3 were labeled Deferiprone. 0.1 mL of the tannic acid and iron(III) chloride were pipetted onto the boiling beads in the vials. After mixing, 9.8 mL of 0.1 M, pH 7 MOPS was added. The vials were left to stand for a week at room temperature to complete the staining. For the destaining phase, the liquid in each vial was discarded and the beads were rinsed with distilled water 3 times. Then the solutions were added to the vials according to Table 1, swirled and left for the destaining phase.

TABLE 1
Solutions added to vials for destaining in Experiment 1.

| Vial | Control | EDTA | C-2 | Deferiprone |
|---|---|---|---|---|
| Solution | 10 mL MOPS | 10 mL 0.1M | 10 mL 0.1M C-2 | 10 mL 0.1M |
| added | | EDTA | | Deferiprone |

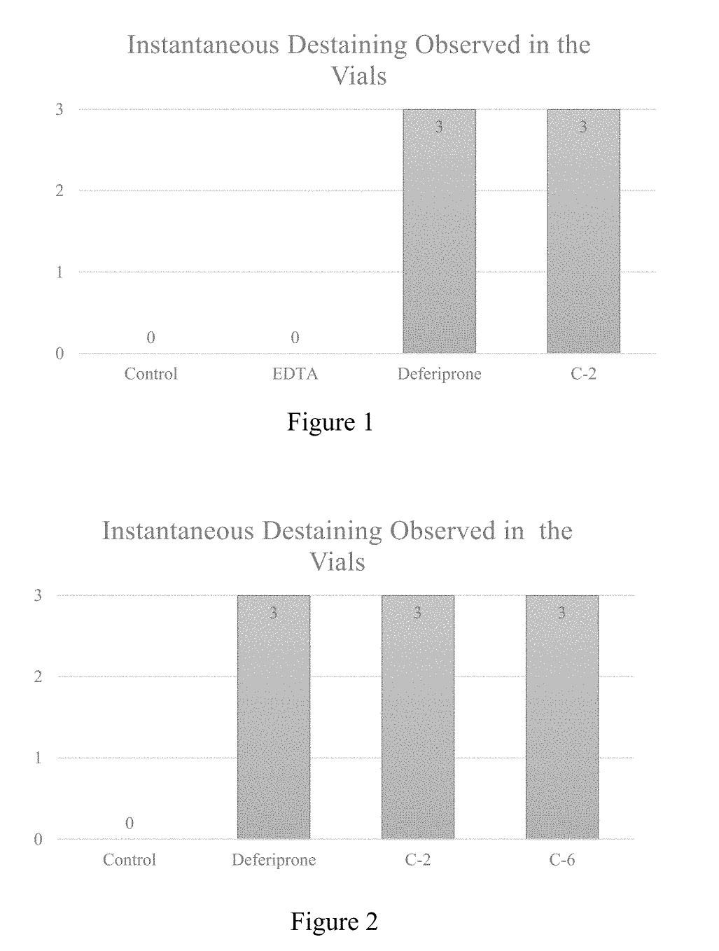

Experiment #2: Comparison of deferiprone, C-2, and C-6 destaining: The same procedure was followed for Experiment 2 except EDTA was replaced with C-6 solution. The results are shown in FIGS. 1 and 2.

TABLE 2
Solutions added to vials for destaining in Experiment 2.

| Vial | Control | C-6 | C-2 | Deferiprone |
|---|---|---|---|---|
| Solution | 10 mL | 10 mL 0.1M C-6 | 10 mL 0.1M C-2 | 10 mL 0.1M |
| added | MOPS | | | Deferiprone |

Experiment #1: Destaining was immediately noted with deferiprone and C-2. EDTA showed a slight difference after 15 minutes but it was not as drastic as the change seen with deferiprone and C-2.

Experiment #2: Destaining was immediately noted in all vials except the control at an equivalent rate.

In conclusion, Deferiprone, C-2, and C-6 are effective in removal of stains and act to remove stains faster than EDTA.

### Example 3

Tannic acid and iron chloride solutions were prepared as in Example 2. In order to simulate a tooth like structure, boiling beads made from a synthetic silicate (Techniservice) and white quartz sand were used. Four vials were prepared with the following solutions and solids (Table 3).

TABLE 3
Vial contents.

| Vial #1 | Vial #2 | Vial #3 | Vial #4 |
|---|---|---|---|
| 2.2002 g of | 2.0489 g of beads | 2.2429 g of sand | 2.0621 g of sand |
| beads | | white quartz | white quartz |

0.1 mL of the tannic acid and iron(III) chloride were pipetted into each vial. They were mixed on a vortex for 10 seconds. Then, 9.8 mL of 0.1 M, pH 7.4 MOPS was added to each vial to adjust the pH to a neutral level. MOPS was used to mimic the pH of saliva. The contents were mixed again. Partial coating of the beads and sand occurred with staining, but they were not completely coated. The vials were left to stand for a week at room temperature. The following table (Table 4) summarizes the components added to the tooth-like solids.

TABLE 4
Summary of components added to the tooth-like solids.

| | | | Volume | Grams | | |
|---|---|---|---|---|---|---|
| | | Concentration | Used from | Used from | | |
| MOPS | | of Stock | the Stock | the Stock | | Final |
| used | Solution | Solution | Solution | Solution | Concentration | Volume |
| 9.8 mL | Tannic Acid | 40 mg/mL | 0.1 mL | 4 mg | 0.4 mg/mL | 10 mL |
| 9.8 mL | Iron (III) Chloride | 10 mg/mL | 0.1 mL | 1 mg | 0.1 mg/mL | 10 mL |

The solids in the vials were washed with approximately 10 mL of MOPS. This step was to simulate a wash of the “tooth” like structure with a “saliva” like buffer. The vials were left for a week to allow time for unstained areas of the beads to stain.

After the structures were stained, experiments were carried out to test if the stains could be removed. Approximately 5 mL of C11 (1-(11′-Carboxydecyl)-3-Hydroxy-2-Methyl-4-Pyridinone) solution was pipetted into Vials #1 and #3. Vials #2 and #4 served as controls. The contents of the vials were then mixed by vortexing. The vials were left to de-stain for a week. After a week, the liquid was drained from the vials and a fresh 5 mL of C11 was pipetted. Vials were left to de-stain for another week.

EDTA was prepared as follows. 100 mM of EDTA was made by adding 9.164 g to 250 mL of deionized water. The solution was stirred for 10 minutes, then placed on a heater. In 15 minutes a homogenous mixture was seen.

Three new vials were filled with 2.2346 g, 2.0195 g, 2.2600 g of beads followed by the salt (0.1 mL), then the tannic acid (0.1 mL), and finally the MOPS. The beads were stained. Three other vials were filled with 2.115 g, 2.009 g, 2.115 g of sand white quartz and the solutions were added in sequence. Once the tannic acid and iron chloride solutions were added, the beads and sand was immediately stained. The solutions were left to continue to stain for a week. After a week, all the vials were rinsed with 10 mL of MOPS. Supernatant was removed by using filter paper. 10 mL of MOPS was then added and the contents were stirred then left to sit for a week.

1 mL of the EDTA was pipetted into each vial. The resulting concentration was 0.01 M of EDTA in each vial. Vials were then mixed to ensure proper contact with the MOPS and EDTA molecules. Vials were left to sit for a week.

The clear solutions of EDTA and MOPS developed a red color after a week, indicating the beads were partially destained. The white quartz sand solutions were clear, but that may be because the amount of stains on the sand was low. In order to validate the tests, the following two controls were run.

Two more pairs of vials (4^{th} and 5^{th} pairs) were prepared—one with the beads and the other with the sand. MOPS solution was added, but not EDTA. The purpose of these controls was to confirm that EDTA alone was involved in destaining. Two more pairs of vials (6^{th} and 7^{th} pairs) were prepared, again, one with beads and the other with sand. The purpose of these controls was to test if the destaining process could be accelerated. The EDTA concentration was increased from 0.01 M to 0.1 M and 10 mL of it was subsequently added to the vials along with 10 mL of MOPS.

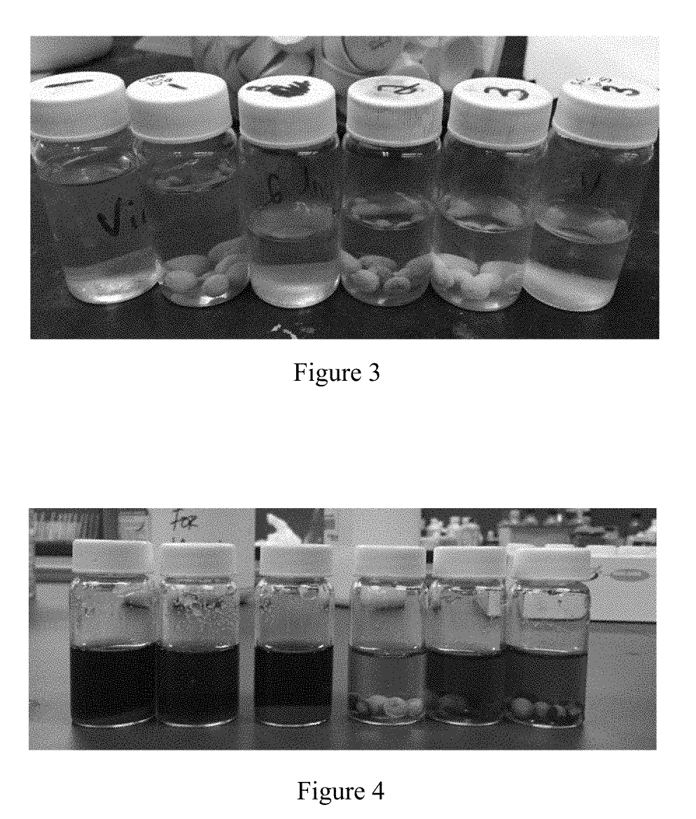

After over a month, the samples were checked and most of the beads and sand were bleached indicating that EDTA removed the tannic acid that was bonded to these materials. FIG. 3 shows the bleached solutions, most of the materials in the solution were bleached.

0.1 M of Deferiprone solution was made by mixing 1.3957 g and 100 mL of distilled water into a volumetric flask. The slow solubility was observed, but at room temperature with a magnetic bar it did dissolve after an entire day of mixing. The tables below (Table 5 and Table 6) summarizes the contents of the vials used in this part of the experiment.

TABLE 5
Summary of contents of the vials used in the experiment.

| | | | Iron (III) | | Distilled |
|---|---|---|---|---|---|
| Vial | Beads | Tannic Acid | Chloride | | Water |
| Label | Used | Used | Used | MOPS used | Used |
| A1 | 2.0106 g | 0.1 mL | 0.1 mL | 5 mL | 5 mL |
| A2 | 2.1334 g | 0.1 mL | 0.1 mL | 9.8 mL | 0 |
| A3 | 2.1735 g | 0.1 mL | 0.1 mL | 9.8 mL | 0 |

TABLE 6
Summary of contents of the vials used in the experiment.

| | | | Iron (III) | |
|---|---|---|---|---|
| | Sand White | Tannic Acid | Chloride | |
| Vial Label | Quartz Used | Used | Used | MOPS used |
| B1 | 2.2728 g | 0.1 mL | 0.1 mL | 9.8 mL |
| B2 | 2.0800 g | 0.1 mL | 0.1 mL | 9.8 mL |
| B3 | 2.0345 g | 0.1 mL | 0.1 mL | 9.8 mL |

FIG. 4 shows the vials. These vials were left to stand for a week. After the week the vials were rinsed with distilled water until the liquid that the beads/sand were suspended in were clear. Controls and non-controls were prepared as shown in the following table (Table 7).

TABLE 7
Identification of vials.

| | Control. 10 mL | | 1 mL of 0.1M |
|---|---|---|---|
| | of | 10 mL of | deferiprone & 9 mL |
| | distilled | 0.1M | of distilled |
| Vial | water | deferiprone | water |
| A1 | Yes | | |
| A2 | | Yes | |
| A3 | | | Yes |
| B1 | Yes | | |
| B2 | | Yes | |
| B3 | | | Yes |

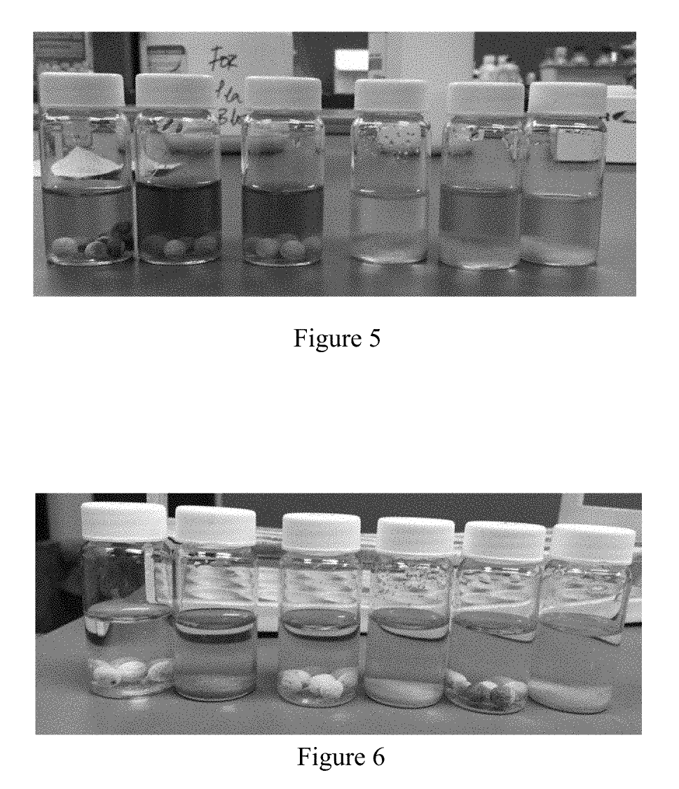

The vials were left again for a week and the table (Table 8) below summarizes the results. FIG. 5 shows the vials for the data from Table 8.

TABLE 8
Results of destaining.

| | Stained |
|---|---|
| Vial | removed? |
| A1 | No |
| A2 | Yes |
| A3 | Yes |
| B1 | No |
| B2 | Yes |
| B3 | Yes |

After Deferiprone was added to vials A2 and B2, there was an immediate discoloration of the solution. Vials A3 and B3 exhibited a relatively slower discoloration.

The next set of experiments was carried out to determine how quickly the compositions were able to remove the stains. The tables below (Table 9 and Table 10) shows the content of the new vials.

TABLE 9
Contents of vials used.

| | | | Iron (III) | |
|---|---|---|---|---|
| | | Tannic Acid | Chloride | Distilled |
| Vial Label | Beads Used | Used | Used | Water Used |
| B1 | 2.0025 g | 0.1 mL | 0.1 mL | 9.8 mL |
| B2 | 2.0864 g | 0.1 mL | 0.1 mL | 9.8 mL |
| B3 | 2.1351 g | 0.1 mL | 0.1 mL | 9.8 mL |
| B4 | 2.0445 g | 0.1 mL | 0.1 mL | 9.8 mL |
| B5 | 2.0092 g | 0.1 mL | 0.1 mL | 9.8 mL |
| B6 | 2.0380 g | 0.1 mL | 0.1 mL | 9.8 mL |

TABLE 10
Contents of vials used.

| | Sand White | | Iron (III) | Distilled |
|---|---|---|---|---|
| Vial Label | Quartz | Tannic Acid | Chloride | Water |
| D1 | 1.9815 g | 0.1 mL | 0.1 mL | 9.8 mL |
| D2 | 2.0249 g | 0.1 mL | 0.1 mL | 9.8 mL |
| D3 | 2.0830 g | 0.1 mL | 0.1 mL | 9.8 mL |
| D4 | 2.0770 g | 0.1 mL | 0.1 mL | 9.8 mL |
| D5 | 2.0661 g | 0.1 mL | 0.1 mL | 9.8 mL |
| D6 | 2.0444 g | 0.1 mL | 0.1 mL | 9.8 mL |

Beads and sand solutions were rinsed with 10 mL of distilled water. The water became colored as it was poured into the vials indicating that the stains that were partially attached to the beads/sand were being removed. The solutions were prepared as follows: (Table 11).

TABLE 11
Identification of vials.

| | | | | 0.1 mL OF 0.1M | 0.01 mL OF 0.1M |
|---|---|---|---|---|---|
| | CONTROL | | 1 mL OF 0.1M | DEFERIPRONE | DEFERIPRONE |
| | (10 mL | 10 mL OF 0.1M | DEFERIPRONE | & 10 mL OF | & 10 mL OF |
| VIAL | H₂O) | DEFERIPRONE | & 9 mL OF H₂O | H₂O | H₂O |
| A2 | Yes | | | | |
| A3 | | Yes | | | |
| A4 | | | Yes | | |
| A5 | | | | Yes | |
| A6 | | | | | Yes |
| B2 | Yes | | | | |
| B3 | | Yes | | | |
| B4 | | | Yes | | |
| B5 | | | | Yes | |
| B6 | | | | | Yes |

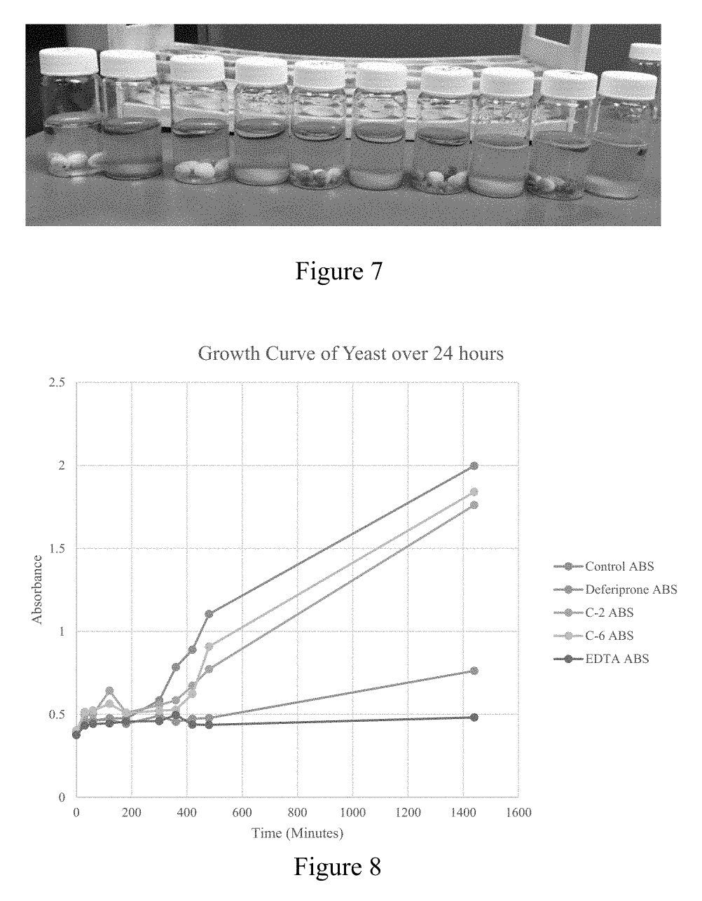

Again, immediately after 10 mL of 0.1M Deferiprone was added to vials A3 and and B3, the stains were removed. For about an hour the destaining for all the vials except the controls continued to be effective but, after the hour, the change was negligible. FIGS. 6 and 7 show the vials.

Of the three compounds (C11, EDTA, and Deferiprone) tested for the destaining that was produced by tannic acid—FeCl₃. Deferiprone was most effective followed by EDTA.

### Example 4

This example shows the antimicrobial activity of compounds of the present disclosure.

This example shows the effect of iron chelators, such as C-2, C-6, and Deferiprone, on yeast in comparison to a control containing no iron chelator and control of EDTA (an iron chelator in which no yeast growth is expected) using liquid yeast, peptone, dextrose (YPD) media to build a growth curve and disk diffusion using YPD agar media. The following materials were used: peptone, dextrose/glucose, marmite/yeast extract, agar, 0.1 M EDTA in MOPS solution, 0.1 M C-2 in MOPS solution, 0.1 M C-6 in MOPS solution, and 0.3 M Deferiprone in MOPS solution, and water.

Plate media was prepared containing 2% dextrose, 2% peptone, 0.36 g of yeast extract, and 1.5% agar in 1 L of water. The mixture was heated to dissolve the ingredients, and then autoclaved. 10 mL of the media was added to petri dishes to prepare plates.

The yeast culture was prepared by adding dry baker's yeast to warm water and streaked on the media plates. Cultures were allowed to grow for 16-24 hours.

The growth curve experiment were carried out by measuring the inhibition of yeast culture in the presence of EDTA, C-2, C-6, and Deferiprone over time via absorbance at 400 nm (see FIG. 8).

The growth curve was used to measure the rate of growth of yeast over an 8 hour period with a final measurement after 24 hours. No growth is seen in the presence of EDTA. C-2, C-6, and Deferiprone show different levels of effectiveness (see FIG. 8).

The results showed that EDTA as a control was successful. EDTA over the 24 hr (hour) period showed least increase in absorbance with initial absorbance at 0.375 and final at 0.481. Deferiprone showed the next best results with initial absorbance at 0.389 and final of 0.762. The control without C-2, C-6, EDTA, or Deferiprone showed an initial absorbance of 0.375 and final of 1.997, which is an increase over both EDTA and Deferiprone. C-2 and C-6 showed final absorbance of 1.761 and 1.840 respectively. C-2 and C-6 were effective, although the effect was less than EDTA and Deferiprone (see FIG. 8).

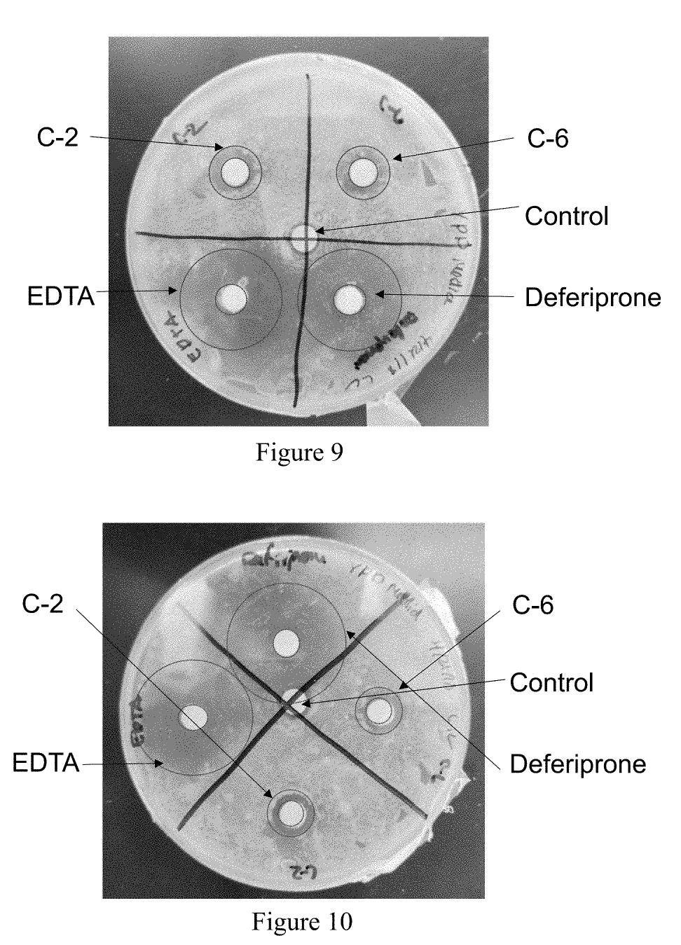

Disks to which C-2, C-6, EDTA, or Deferiprone were added were added to petri dishes containing yeast cultures. The plates were incubated at 30° C. The effect on yeast growth was visualized and measured over time. The zone sizes were measured to the nearest millimeter using a ruler or caliper, including the diameter of the disk in the measurement. The measurements were rounded up to the nearest millimeter. Disk diffusion showed similar results to the growth curve. With EDTA and Deferiprone being the most effective, with diameters larger than 2.45 cm. C-2 and C-6 showed diameters of 1.55 cm and 1.45 cm, respectively (see FIGS. 9 and 10).

These data indicate the compounds of the present disclosure have antimicrobial properties.

Although the present disclosure has been described with respect to one or more particular embodiments and/or examples, it will be understood that other embodiments and/or examples of the present disclosure may be made without departing from the scope of the present disclosure.

What is claimed:

1. An oral formulation comprising one or more compounds having the structure of: 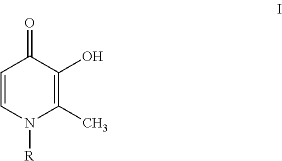
where R is selected from the group consisting of 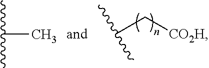
where n=1 to 11, in an orally acceptable carrier.

2. The oral formulation of claim 1, wherein the compound of Structure I is present at a concentration of from 0.001 molar to 1.0 molar.

3. The oral formulation of claim 1, wherein R is —CH₃.

4. The oral formulation of claim 1, wherein R is 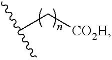
and n=1, 5, 10 or 11.

5. The oral formulation of claim 1, wherein the formulation is in the form of a gel, paste, powder, foam, or liquid.

6. The oral formulation of claim 1, wherein the formulation is incorporated into a strip.

7. A method of removing stains from a tooth comprising contacting the stained tooth with a composition of claim 1.
